(19) United States
(12) Patent Application Publication
(10) Pub. No.: US 2011/0034739 A1
(43) Pub. Date: Feb. 10, 2011

(54) CATALYST AND PROCESS FOR PREPARING SATURATED ETHERS BY HYDROGENATING UNSATURATED ETHERS

(75) Inventors: Guido Stochniol, Haltern am See (DE); Silvia Santiago Fernandez, Madrid (ES); Franz Nierlich, Marl (DE); Stephan Houbrechts, Duffel (BE); Wilfried Bueschken, Haltern am See (DE)

Correspondence Address: OBLON, SPIVAK, MCCLELLAND MAIER & NEUSTADT, L.L.P., 1940 DUKE STREET, ALEXANDRIA, VA, 22314, US

(73) Assignee: EVONIK OXENO GMBH, Marl (DE)

(21) Appl. No.: 12/937,657

(22) PCT Filed: May 7, 2009
(86) PCT No.: PCT/EP2009/055512

§ 371 Date: Oct. 13, 2010

(30) Foreign Application Priority Data
Jun. 11, 2008 (DE) 102008002347.7

(51) Int. Cl. B01J 27/25; B01J 37/02; B01J 37/08; C07C 41/20

(52) U.S. Cl. 568/671; 502/201

(57) ABSTRACT

The invention relates to a supported catalyst based on palladium-γ-alumina, which is characterized in that the catalyst support material contains 1 to 1000 ppm by mass of sodium oxide and has a specific pore volume of 0.4 to 0.9 ml/g and a BET surface area of 150 to 350 m²/g. The invention further relates to a process for hydrogenating polyunsaturated ethers with hydrogen to the corresponding saturated ethers, in which the catalyst used is such a catalyst.

The present invention relates to a catalyst and to a process for preparing saturated ethers by hydrogenating unsaturated ethers, especially for preparing alkoxyoctanes and alkoxydimethyloctanes by hydrogenating octadienyl alkyl ethers or dimethyloctadienyl alkyl ether.

Alkoxy compounds of octanes or dimethyloctanes are precursors for the preparation of octenes or dimethyloctenes. 1-Alkoxyoctane can be used, for example, as a precursor for the preparation of 1-octene, which is used as a comonomer to modify polyethylene and polypropylene. It is known that octadienyl alkyl ethers and dimethyloctadienyl alkyl ethers can be prepared by reacting 1,3-butadiene or isoprene with alcohols (telomerization).

The hydrogenation of the unsaturated ethers mentioned to the corresponding saturated ethers is known per se. WO 2005/019139 describes the hydrogenation of octadienyl ethers to the corresponding saturated ethers, more particularly the hydrogenation of 1-methoxy-2,7-octadiene. The hydrogenation is performed in the presence of a supported catalyst consisting of 5% by mass of palladium on barium sulphate in the temperature range of 0 to 100° C. and in a pressure range of 1 to 25 bar. The description states that, in the hydrogenation, it is possible to use solvents, for example ethers, aromatic hydrocarbons, paraffins, halogenated hydrocarbons and nitriles. In the examples, the hydrogenation is performed without use of a solvent.

EP 0 561 779 describes a process for hydrogenating octadienyl ethers, in which the hydrogenation catalysts used include supported catalysts consisting of 0.1 to 10% by mass of palladium on γ-alumina. The hydrogenation is performed in the temperature range of 50 to 200° C. and in the pressure range of 0.1 to 100 bar. Optionally, the hydrogenation can be performed in the presence of a solvent, for example of an alcohol. In the example, 99.3% 1-methoxy-2,7-octadiene is hydrogenated at 80° C. and 15 bar over a supported catalyst consisting of 0.3% by mass of palladium on γ-alumina with hydrogen in the absence of a solvent. The yield of saturated ether is virtually 100%. No details of the catalyst are given, and so it has to be assumed that all such palladium-γ-alumina supported catalysts are suitable for the hydrogenation of alkoxyoctadienyl ethers to the corresponding saturated ethers.

The preparation of alkoxyoctadienyl derivatives by reaction of butadiene or isoprene with an alcohol (telomerization) forms product mixtures which still contain the alcohol used. The full depletion of the excess alcohol is often complicated. When octenes or dimethyloctenes are to be prepared from the telomerization product anyway by hydrogenation of the two olefinic double bonds and subsequent alcohol elimination, a portion of the alcohol present in the telomerization product and the alcohol formed in the cleavage of the hydrogenation product can be removed in a combined workup step. However, this method is advantageous only when no by-products form from the alcohol during the hydrogenation. Otherwise, the formation of by-products, for example the formation of dimethyl ether from methanol, would increase not just the workup complexity but also the specific material costs.

The known supported catalysts which have been used to date in such processes additionally have the disadvantage that their activity declines relatively rapidly and they thus do not have sufficiently long-lasting long-term activity.

It was therefore an object of the present invention to provide a catalyst with which octadienyl ether mixtures can be hydrogenated to the corresponding octyl ethers with hydrogen without formation of by-products in the presence of alcohol, and which more particularly has a high long-term activity.

It has now been found that supported catalysts based on palladium-γ-alumina are particularly suitable for the selective hydrogenation of polyunsaturated ethers, especially octadienyl ethers and mixtures thereof, to the corresponding saturated ethers, especially octyl ethers, when the catalyst support material contains 1 to 1000 ppm by mass of sodium oxide and has a specific pore volume of 0.4 to 0.9 ml/g and a BET surface area of 150 to 350 m²/g.

The present invention therefore provides a supported catalyst based on palladium-γ-alumina, which is characterized in that the catalyst support material contains 1 to 1000 ppm by mass of sodium oxide and has a specific pore volume of 0.4 to 0.9 ml/g and a BET surface area of 150 to 350 m²/g, and also a process for preparing it.

The present invention further provides a process for preparing saturated ethers by hydrogenating unsaturated ethers, in which the catalyst used is a supported catalyst based on palladium-γ-alumina, which is characterized in that the catalyst support material contains 1 to 1000 ppm by mass of sodium oxide and has a specific pore volume of 0.4 to 0.9 ml/g and a BET surface area of 150 to 350 m²/g.

The present invention exhibits the following unexpected advantages:

The hydrogenation of octadienyl ethers to the corresponding saturated ethers is not disrupted by accompanying substances. For instance, the inventive catalyst does not promote the formation of ethers as a result of elimination of water from any alcohols present in the hydrogenation. Any high boilers present in the mixture in small concentrations do not cause any significant deterioration in the hydrogenation activity of the catalyst. A particular advantage of the invention is that the catalyst has a long service life and the hydrogenation selectivity during the run time remains virtually constant. This is surprising in particular because, as Example 2 shows, customary palladium-γ-alumina catalysts do not provide this performance.

The process according to the invention and the catalysts according to the invention are described in detail below.

The inventive supported catalyst based on palladium-γ-alumina is characterized in that the parent γ-alumina support material contains 1 to 1000 ppm by mass of sodium oxide and has a specific pore volume of 0.4 to 0.9 ml/g and a BET surface area of 150 to 350 m²/g.

To prepare the inventive supported catalyst, a support material based on γ-alumina is used, which contains 1 to 1000 ppm by mass of sodium compounds (calculated as sodium oxide). The support material preferably contains 1 to 750 ppm by mass, especially 1 to 500 ppm by mass, of sodium compounds (calculated in each case as sodium oxide).

Optionally, the support material may contain sulphate or sulphate groups and/or silicon dioxide. The sulphate content may be up to 1500 ppm by mass. In addition, the support material may contain up to 20% by mass of silica.

The BET surface area of the support material used is 150 to 350 m²/g, preferably 200 to 320 m²/g, more preferably 220 to 300 m²/g (determined by the BET method by nitrogen adsorption to DIN 9277).

The pore volume of the support material is 0.4 to 0.9 ml/g (determined by mercury intrusion to DIN 66133).

The mean pore radius of the support material is preferably 2 to 50 nm, more preferably 5 to 30 nm and especially 7 to 15 nm (determined by combining the pore size distribution to DIN 66133 and determining the mesopores according to BJH to DIN 66134).

Suitable γ-alumina support materials of this type are commercially available from many sources.

The inventive supported catalyst contains palladium as the hydrogenation-active component. The palladium content in the ready-to-use catalyst is preferably 0.1 to 10% by mass, especially 0.1 to 3% by mass and more preferably 0.2 to 1% by mass.

The inventive catalyst can be prepared by applying one or more palladium compound(s) to a support material as described above. The application can be effected by impregnating the support with a solution containing palladium compound, spray application of solutions containing palladium compounds to the support, or by other methods with like effect. Suitable palladium compounds which can be applied to the support are, for example, palladium acetate, palladium acetylacetonate, palladium chloride, palladium nitrate dihydrate or palladium sulphate dihydrate, palladium nitrate dihydrate being the preferred compound. The solutions comprising palladium compounds used are preferably aqueous palladium salt solutions. Such solutions preferably have a palladium content of 1 to 15% by mass, preferably of 5 to 10% by mass.

After the application of the palladium compound(s), the support material is dried, typically at temperatures of 80 to 150° C., and optionally calcined at temperatures of 200 to 600° C.

In a particular embodiment, the application of the palladium compound(s), drying and optional calcinations can be effected in one step. For instance, the inventive supported catalyst can be obtained by spray application of a solution of a palladium compound to the support material at a temperature of 80° C. or higher.

The inventive supported catalysts are preferably prepared by spray application of an aqueous solution comprising palladium salt compounds to the support material at temperatures of 10 to 170° C., especially of 50 to 150° C., and optional subsequent calcination in the temperature range of 170 to 550° C., especially of 200 to 450° C. When the spray application is undertaken at standard pressure, the temperature of the material to be sprayed is preferably 100 to 170° C. When the spray application is performed under reduced pressure, the pressure preferably being less than the partial water vapour pressure of the spray solution, the temperature is preferably 20 to 100° C.

In the course of spray application, the majority of the water present in the spray solution evaporates. This achieves the effect that the palladium is present on the support material in a boundary layer which encompasses a thickness of 50 to 300 μm. Typically, about 90% of the palladium applied is within this boundary layer.

The inventive supported catalysts are preferably prepared in a form which offers low flow resistance in the course of hydrogenation. Typical forms are, for instance, tablets, cylinders, extrudates or rings. The shaping is generally effected on the support material before the application of the palladium compound. It is also possible to use granulated supports to produce the supported catalysts. Screening allows a catalyst support with the desired particle size to be removed. Frequently, γ-alumina or support materials containing γ-alumina can actually be purchased in the form of corresponding shaped bodies.

The process according to the invention for preparing saturated ethers by hydrogenating unsaturated ethers is notable in that the catalyst used is a supported catalyst which is based on palladium-γ-alumina and is characterized as above. In the process according to the invention, it is possible to use mixtures which contain polyunsaturated ethers and alcohol, preferably methanol, ethanol and/or propanol. The molar ratio of alcohol to polyunsaturated ether in the reactant mixture is typically 2:98 to 40:60, especially 5:95 to 25:75, and more preferably 10:90 to 22:78.

The process can be performed continuously or batchwise. Preference is given to performing the process continuously. The hydrogenation can be performed over inventive supported catalysts arranged in a fixed bed.

In the process according to the invention, the hydrogenation can be performed in the liquid phase or in the gas phase. In the process according to the invention, preference is given to performing a continuous hydrogenation over an inventive supported catalyst arranged in a fixed bed, in which the product/reactant phase is present principally in the liquid state under reaction conditions.

When the hydrogenation is performed continuously over a catalyst arranged in a fixed bed, it is appropriate to convert the supported catalyst to the active form before the hydrogenation. This can be done by reducing the supported catalyst with hydrogenous gases using a temperature programme. For example, the catalyst is heated up to 200° C. at 5 K/min in an H₂ stream, and the temperature is maintained for 2 h and then lowered to reaction temperature. The reduction can optionally be performed in the presence of a liquid phase which trickles over the catalyst. The liquid phase used may be a solvent or preferably the hydrogenation product.

For the process according to the invention, different process variants can be selected. It can be performed adiabatically, polytropically or virtually isothermally, i.e. with a temperature rise of typically less than 10° C., and in one or more stages. In the latter case, it is possible to operate all reactors, preferably tubular reactors, adiabatically or virtually isothermally, or else one or more adiabatically and the others virtually isothermally. In addition, it is possible to hydrogenate the saturated compounds in straight pass or with product recycling.

The process according to the invention is preferably performed in the liquid/gas mixed phase or liquid phase in triphasic reactors in cocurrent, the hydrogenation gas being distributed within the liquid reactant/product stream in a manner known per se. In the interests of homogeneous liquid distribution, of improved removal of heat of reaction and of a high space-time yield, the reactors are usually operated with high liquid velocities of 15 to 120 m³, especially of 25 to 80 m³, per m² of cross section of the empty reactor and hour. When a reactor is operated in straight pass, the specific liquid hourly space velocity (LHSV) may assume values between 0.1 and 10 h⁻¹.

The hydrogenation can be performed in the absence or in the presence of a solvent. Preference is given to performing the hydrogenation in the presence of a solvent. The use of a solvent allows the concentration of the polyunsaturated ether to be hydrogenated in the reactor feed to be limited, which allows better temperature control in the reactor to be achieved. In this way, minimization of side reactions and hence an increase in the product yield are achieved. Preference is given to adjusting the concentration of the polyunsaturated ether to be hydrogenated in the reactor feed, and in the case of a plurality of reactors more particularly in the feed to the first reactor, to a concentration of 1 to 35% by mass, more preferably 5 to 25% by mass. The desired concentration of the polyunsaturated ether to be hydrogenated in the reactor feed can, in the case of reactors operated in loop mode, be established through the circulation ratio (quantitative ratio of hydrogenation effluent recycled to reactant).

The solvents used may be all liquids which form a homogeneous solution with the reactant and product, behave inertly under hydrogenation conditions and can be removed easily from the product. The solvent may also be a mixture of several substances and may optionally contain water. The solvent used is preferably a saturated ether, as obtained, for instance, as a hydrogenation product in the process according to the invention. In this way, it is possible to avoid a complicated step in which the solvent is removed again from the product discharge.

The process according to the invention is preferably performed at a pressure of from 20 to 150 bar, preferably at 30 to 120 bar and more preferably at 40 to 100 bar. The hydrogenation temperature at which the process is performed is preferably 50 to 150° C., especially 60 to 120° C.

The hydrogenation gases used may be hydrogen or any hydrogenous gases or gas mixtures. The gases used should not contain any harmful amounts of catalyst poisons, for example carbon monoxide or hydrogen sulphide. Preference is given to using gases which contain neither carbon monoxide nor hydrogen sulphide. In addition to hydrogen, the gases used may contain one or more inert gas(es). Inert gas constituents may, for example, be nitrogen or methane. The hydrogenous gas used is preferably hydrogen in a purity of greater than 95% by volume, especially of greater than 98% by volume.

Hydrogen is used in a stoichiometric excess. The excess is preferably more than 10%.

By means of the process according to the invention, polyunsaturated ethers can be hydrogenated to the corresponding saturated ethers. Preference is given to using the process according to the invention to hydrogenate alkyl-substituted or unsubstituted octadienyl alkyl ethers to the corresponding alkyl-substituted or unsubstituted saturated octyl alkyl ethers. The alkyl group may, for example, be a methyl, ethyl or propyl group. The alkyl group is more preferably a methyl group. Very particular preference is given to using the process according to the invention to hydrogenate 1-methoxy-2,7-octadiene to 1-methoxyoctane.

The feedstocks mentioned can be obtained, for example, by telomerization. In the telomerization, two moles of diene are reacted with one mole of alcohol. The telomerization of isoprene forms dimethyloctadienyl alkyl ethers, the telomerization of butadiene forms octadienyl alkyl ethers, and the crossed telomerization of isoprene and butadiene forms a mixture of dimethyloctadienyl alkyl ethers, methyloctadienyl alkyl ethers and octadienyl alkyl ethers. The alcohols used in the telomerization may especially be methanol, ethanol or propanol. In the telomerization, methanol is preferably used as the alcohol.

Preferred feedstocks are alkyl-substituted or unsubstituted octadienes with a terminal alkoxy group, especially a methoxy group. A very particularly preferred feedstock is 1-methoxyocta-2,7-diene. Processes for preparing this compound are described, for example, in DE 101 49 348, DE 103 12 829, DE 10 2005 036039.4, DE 10 2005 036038.6, DE 10 2005 036040.8.

The feedstocks used for the inventive hydrogenation need not be pure substances, but rather may also contain further components. For example, 1-methoxyocta-2,7-diene (1-MODE) prepared by telomerization frequently contains a few percent by mass of 3-methoxyocta-2,7-diene. In addition, it is also possible for other double bond isomers to be present. Technical mixtures may additionally contain methanol, solvents and by-products from the telomerization.

The reaction mixtures obtained in the inventive hydrogenation can be used as such or worked up, for example by distillation.

Monoolefins can be obtained by alcohol elimination from the saturated ethers prepared by hydrogenation. For example, 1-methoxyoctane (1-MOAN) can be converted to 1-octene. Such a process is described, for instance, in DE 102 57 499.

The examples which follow are intended to illustrate the invention without limiting it to them.

### EXAMPLE 1

### Preparation of a Palladium-γ-Alumina Catalyst (Noninventive)

An alumina support (CPN from Alcoa) was sprayed with a palladium nitrate-containing aqueous solution (Pd content 15% by mass) and then dried at 120° C. for 2 h. This was followed by reduction in a hydrogen-containing nitrogen stream at 200° C. for 2 h. The alumina support consisted of a granule having a mean particle size of 1.2 to 2.4 mm (determined by screen analysis) and had a BET surface area of approx. 250 m²/g, a pore volume of 0.33 ml/g and a sodium oxide content of 0.5% by mass (each manufacturer's data). The penetration depth of the deposited Pd was (according to EDX analysis) approx. 100 to 250 μm. The palladium content based on the total catalyst mass was approx. 0.5% by mass.

### EXAMPLE 2

### Hydrogenation of 1-Methoxy-2,7-Octadiene (MODE) (Noninventive)

I.) In a stirred tank autoclave with a reaction volume of 1.4 l, 60 g of the catalyst were introduced in a basket. The autoclave was filled with 1.4 l of a mixture of 80% by mass of MODE and 20% by mass of methanol. After inertization with nitrogen, the reactor was heated to 80° C. and then brought to a pressure of 15 bar absolute with hydrogen. To start the reaction, the sparging stirrer was set to a rotation of 1000 min⁻¹. To observe the course of the reaction, samples were taken at regular intervals and analysed by gas chromatograph.

After a reaction time of 2 h, the conversion was complete. The content of the 1-MOAN product was 77.5 GC area %. Subsequently, the autoclave was emptied; the catalyst was left in the reactor. The run time of the catalyst was a total of 7 h.

II.) The catalyst with a run time of 7 h was left in the autoclave after test example 2.1. The autoclave was filled with 1.41 of a mixture of 98% by mass of MODE and 2% by mass of methanol. After inertization with nitrogen, the reactor was heated to 80° C. and then brought to a pressure of 15 bar absolute with hydrogen. To start the reaction, the sparging stirrer was set to a rotation of 1000 min⁻¹. To observe the course of the reaction, samples were taken at regular intervals and analysed by gas chromatograph.

After a reaction time of 4 h, the concentration of 1-MOAN was 94.5 GC area %. Subsequently, the autoclave was emptied; the catalyst was left in the reactor.

III.) Repetition of test I with the catalyst used in tests I and II after a total run time of 11 h. The autoclave was filled with 1.4 l of a mixture of 80% by mass of MODE and 20% by mass of methanol. After inertization with nitrogen, the reactor was heated to 80° C. and then brought to a pressure of 15 bar absolute with hydrogen. To start the reaction, the sparging stirrer was set to a rotation of 1000 min⁻¹. To observe the course of the reaction, samples were taken at regular intervals and analysed by gas chromatograph.

After a reaction time of 4 h, the conversion was incomplete. The content of the 1-MOAN product was 42.5 GC area %. The comparison of tests I and III showed a significant decline in the MOAN formation as a result of catalyst deactivation.

### EXAMPLE 3

### Preparation of a Palladium-γ-Alumina Catalyst (Inventive)

An alumina support (SP 538 E, from Axens) was sprayed with a palladium nitrate-containing aqueous solution (Pd content 15% by mass) at 100° C. and then heat-treated at 450° C. for 60 min. For activation, a reduction was effected in a hydrogen stream at 250° C. over 2 h.

The alumina support consisted of an extrudate in the form of cylinders with a diameter of 1.2 mm and lengths which were between 2 and 6 mm, and had a BET surface area of approx. 280 m²/g (determined by the BET method by nitrogen adsorption to DIN 9277), a pore volume of 0.72 ml/g (supplier data), a sodium oxide content of 0.03% by mass (supplier data) and a sulphate content of approx. 0.1% by mass (supplier data). The penetration depth of the deposited Pd was approx. 80 to 150 μm and the palladium content was, based on the total catalyst mass, approx. 0.5% by mass (determined in each case by means of EDX analysis in a study of the catalyst grain cross section with a scanning electron microscope).

### EXAMPLE 4

### Hydrogenation of 1-Methoxy-2,7-Octadiene (Mode) (Inventive)

I.) In a stirred tank autoclave with a reaction volume of 1.4 l, 60 g of the inventive catalyst (from Example 3) were introduced in a basket. The autoclave was filled with 1.4 l of a mixture of 80% by mass of MODE and 20% by mass of methanol. After inertization with nitrogen, the reactor was heated to 80° C. and then brought to a pressure of 15 bar absolute with hydrogen. To start the reaction, the sparging stirrer was set to a rotation of 1000 min⁻¹. To observe the course of the reaction, samples were taken at regular intervals and analysed by gas chromatograph.

After a reaction time of 2 h, the conversion was complete. The content of the 1-MOAN product was 76.3 GC area %. Subsequently, the autoclave was emptied; the catalyst was left in the reactor. The run time of the catalyst was a total of 7 h.

II.) The catalyst with a run time of 7 h was left in the autoclave after test example 2.1. The autoclave was filled with 1.4 l of a mixture of 98% by mass of MODE and 2% by mass of methanol. After inertization with nitrogen, the reactor was heated to 80° C. and then brought to a pressure of 15 bar absolute with hydrogen. To start the reaction, the sparging stirrer was set to a rotation of 1000 min⁻¹. To observe the course of the reaction, samples were taken at regular intervals and analysed by gas chromatograph.

After a reaction time of 2 h, the concentration of 1-MOAN was 93.7 GC area % and remained unchanged even after 4 h. Subsequently, the autoclave was emptied; the catalyst was left in the reactor The run time in this experiment was 4 h.

III.) The test according to I.) was repeated using the catalyst used in I. and II. The total run time of the catalyst up to the start of the test was 11 h. In a stirred tank autoclave with a reaction volume of 1.4 l, 60 g of the catalyst used in I. and II. were introduced in a basket. The autoclave was filled with 1.4 l of a mixture of 80% by mass of MODE and 20% by mass of methanol. After inertization with nitrogen, the reactor was heated to 80° C. and then brought to a pressure of 15 bar absolute with hydrogen. To start the reaction, the sparging stirrer was set to a rotation of 1000 min⁻¹. To observe the course of the reaction, samples were taken at regular intervals and analysed by gas chromatograph.

After a reaction time of 2 h, the conversion was complete. The content of the 1-MOAN product was 72.5 GC area %.

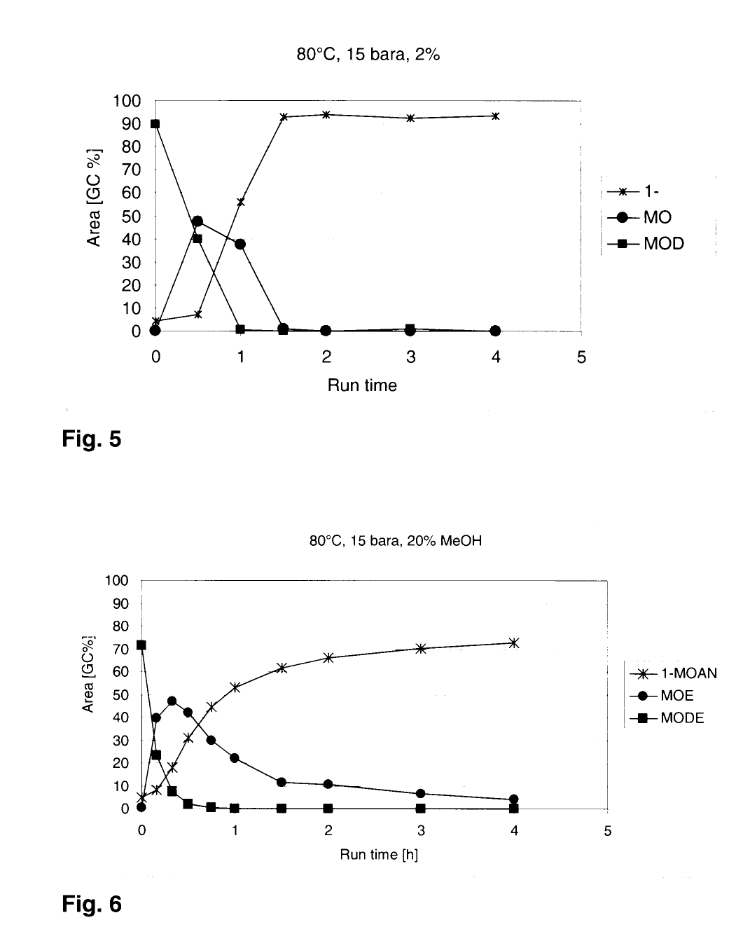

At low MeOH concentrations, the inventive catalyst exhibited a considerably higher hydrogenation performance than the noninventive catalyst (comparison: FIG. 2 and FIG. 5). In addition, the inventive catalyst in the repeat test exhibited significantly lower ageing than the noninventive catalyst (comparison: FIG. 3 and FIG. 6).

### EXAMPLE 5

### Long-Term Test 1 (Inventive)

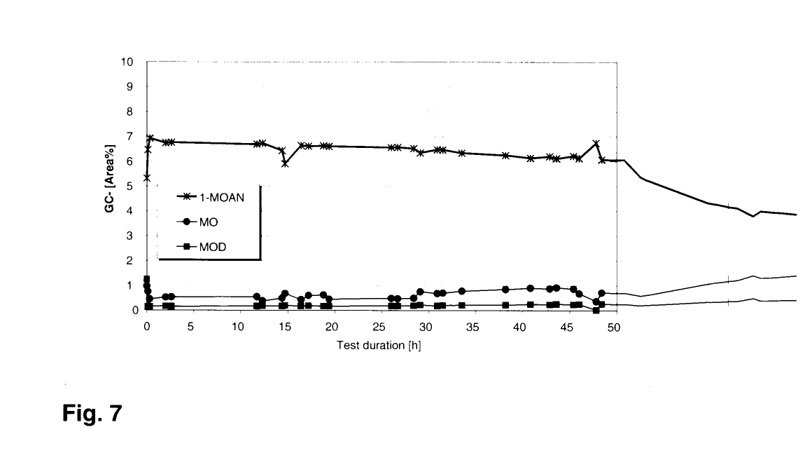

25 g of the inventive catalyst according to Example 3 were placed in a tubular reactor which was part of a circulating hydrogenation apparatus. The reactor was heated to 90° C. and the catalyst was reduced with hydrogen for 2 h. Subsequently, 989 g (1200 ml) of a methanolic MODE solution were charged into the apparatus. The methanol concentration was 20% by mass. Subsequently, hydrogenation was effected at 90° C. and 15 bara. 70 g/h of the feed solution were metered in and a corresponding amount was discharged while keeping the reactor volume constant. After 20 h, the quasi-steady state was attained. The MOAN concentration at the reactor outlet was 67%. After 500 h, the MOAN concentration was still 61%. The changes in the proportions of GC area over the test duration are shown in FIG. 7.

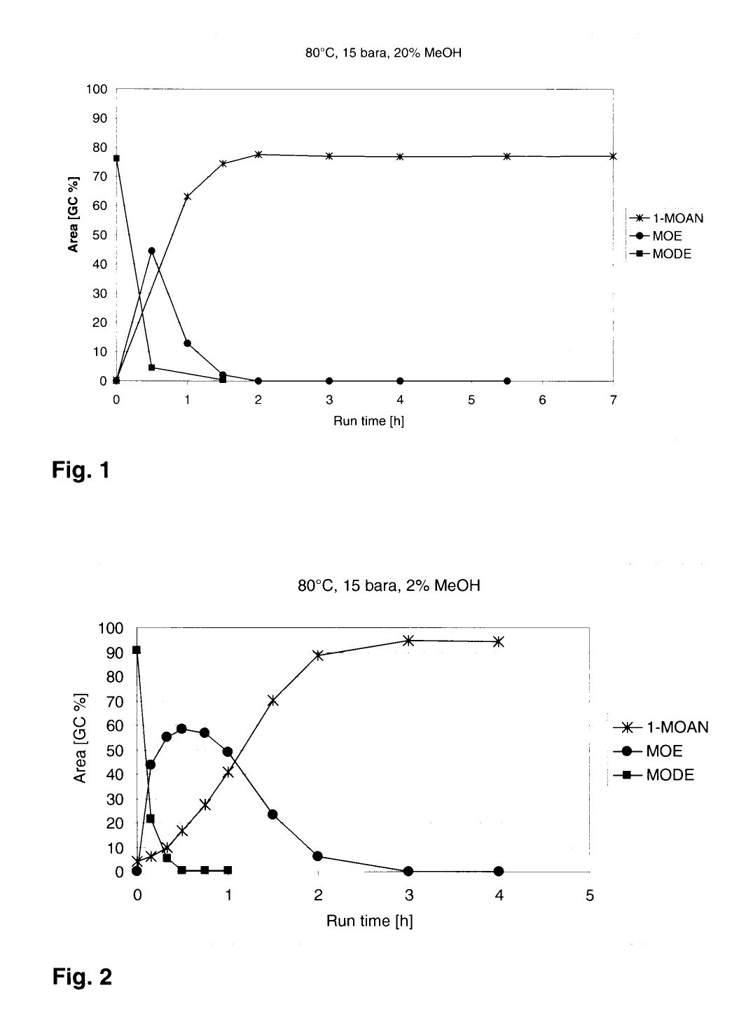

FIG. 1 to FIG. 7 show diagrams in which the course of the GC areas over the reaction times is shown.

In the diagrams:
MODE: 1-methoxyocta-2,7-diene
MOE: 1-methoxyoctene (sum of the individual isomers which differ by the position and/or configuration of the double bond)
MOAN: 1-methoxyoctane

FIG. 1 shows the course of the GC area ratios in the test according to Example 2, I.).

FIG. 2 shows the course of the GC area ratios in the test according to Example 2, II.).

FIG. 3 shows the course of the GC area ratios in the test according to Example 2, III.).

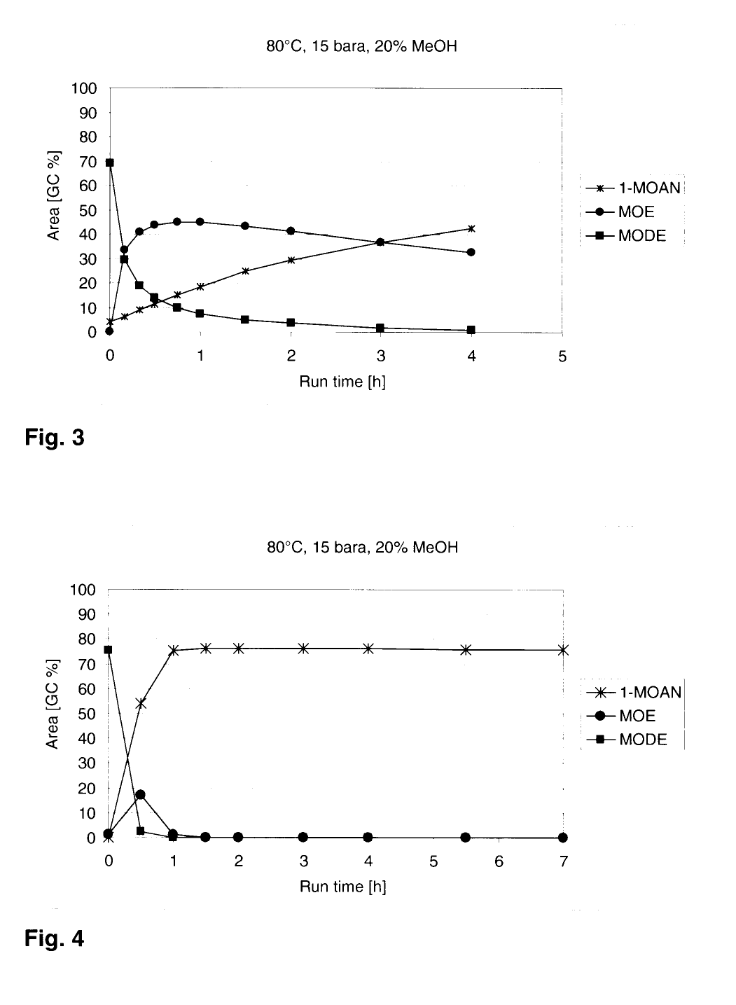

FIG. 4 shows the course of the GC area ratios in the test according to Example 4, I.).

FIG. 5 shows the course of the GC area ratios in the test according to Example 4, II.).

FIG. 6 shows the course of the GC area ratios in the test according to Example 4, III.).

FIG. 7 shows the course of the GC area ratios in the test according to Example 5).

1: A supported catalyst, comprising palladium-γ-alumina and a catalyst support material,
wherein the catalyst support material:
comprises 1 to 1000 ppm by mass of sodium oxide;
has a specific pore volume of 0.4 to 0.9 ml/g; and
has a BET surface area of 150 to 350 m²/g.

2: The supported catalyst according to claim 1, wherein the catalyst support material has a BET surface area of 200 to 320 m²/g.

3: The supported catalyst according to claim 1, wherein palladium content is 0.1 to 10% by mass.

4: The supported catalyst according to claim 1, wherein the palladium palladium-γ-alumina is present on the support material in a boundary layer which has a thickness of 50 to 300 μm.

5: The supported catalyst according to claim 1, wherein a mean pore radius of the support material is 2 to 50 nm.

6: The supported catalyst according to claim 5, wherein the mean pore radius of the support material is 5 to 30 nm.

7: The supported catalyst according to claim 1, wherein the support material comprises at least one selected from the group consisting of sulphate and silicon dioxide.

8: A process for preparing a supported catalyst according to claim 1, comprising
applying a solution comprising a palladium compound to a support material which comprises 1 to 1000 ppm by mass of sodium oxide and has a specific pore volume of 0.4 to 0.9 ml/g and a BET surface area of 150 to 350 m²/g to give a treated support material, and then
drying the treated support material at a temperature of 80 to 150° C.

9: The process according to claim 8, wherein the solution comprising the palladium compound is sprayed onto the support material at a temperature of 80° C. or higher.

10: The process according to claim 8, wherein the palladium compound is palladium acetate, palladium acetylacetonate, palladium chloride, palladium nitrate dihydrate, or palladium sulphate dihydrate.

11: A process for hydrogenating a polyunsaturated ether with hydrogen to give a corresponding saturated ether, the process comprising:
hydrogenating a polyunsaturated ether with hydrogen in the presence of the supported catalyst according to claim 1,
to give the corresponding saturated ether.

12: The process according to claim 11, wherein hydrogenating is performed at a temperature of 50 to 150° C.

13: The process according to claim 11, wherein the hydrogenating is performed at a pressure of 20 to 150 bar.

14: The process according to claim 11, wherein the hydrogenating is performed in the presence of a solvent.

15: The process according to claim 14, wherein the solvent is a saturated ether obtained in the hydrogenating.

16: The process according to claim 11, wherein a concentration of the polyunsaturated ether to be hydrogenated is in a reactor feed and is adjusted to 1 to 35% by mass.

17: The process according to claim 11, wherein the polyunsaturated ether is at least one selected from the group consisting of unsubstituted and alkyl-substituted octadienyl alkyl ethers, which is hydrogenated to the corresponding saturated ether in the hydrogenating.

18: The process according to claim 17, wherein 1-methoxy-2,7-octadiene is hydrogenated to 1-methoxyoctane in the hydrogenating.

19: The supported catalyst according to claim 1, wherein the catalyst support material has a BET surface area of 220 to 300 m²/g.

20: The supported catalyst according to claim 2, wherein palladium content is 0.1 to 10% by mass.
